SEQUENCE LISTING (25 sequences)

SEQ ID NO: 1 - DNA, 22 bp, artificial
  primer
ggctcgagcatggagatacacc

SEQ ID NO: 2 - DNA, 28 bp, Artificial
  primer
ggggactagtttatggtttctgagaaca

SEQ ID NO: 3 - DNA, 31 bp, Artificial
  primer
gggggctagccctcctttgattagtatattc

SEQ ID NO: 4 - DNA, 28 bp, Artificial
  primer
ctccctcgagatcataatttacttcatc

SEQ ID NO: 5 - DNA, 55 bp, Artificial
  primer
gactacaaggacgatgaccgacaagtgataacccgggatctaaataaatccgttt

SEQ ID NO: 6 - DNA, 27 bp, Artificial
  primer
cccgtcgaccagctcttcttggtgaag

SEQ ID NO: 7 - DNA, 156 bp, Listeria monocytogenes
caaatagttggtatagtcctctttagcctttggagtattatctcatcatttgttttttaggtgaaaactgggtaaacttagtattatcaatataaaattaattctcaaatacttaattacgtactgggattttctgaaaaaagagaggagttttcc

SEQ ID NO: 8 - DNA, 21 bp, Artificial
  primer
ccatggtgacaggctggcatc

SEQ ID NO: 9 - DNA, 27 bp, Artificial
  primer
gctagcctaatggatgtattttctagg

SEQ ID NO: 10 - DNA, 25 bp, Artificial
  primer
ggcgccactaactcaacgctagtag

SEQ ID NO: 11 - DNA, 27 bp, Artificial
  primer
gctagccagcaaagaaaaacaaacacg

SEQ ID NO: 12 - DNA, 29 bp, Artificial
  primer
atgaaaaaaataatgctagtttttattac

SEQ ID NO: 13 - DNA, 48 bp, Artificial
  primer
gcggccgcttaatgatgatgatgatgatgtggtttctgagaacagatg

SEQ ID NO: 14 - DNA, 22 bp, Artificial
  primer
gcaagtgtgactctacgcttcg

SEQ ID NO: 15 - DNA, 21 bp, Artificial
  primer
tgcccattaacaggtcttcca

SEQ ID NO: 16 - DNA, 32 bp, Artificial
  primer
tgcgtacaaagcacacacgtagacattcgtac

SEQ ID NO: 17 - DNA, 24 bp, Artificial
  primer
tgacatcgtttgtgtttgagctag

SEQ ID NO: 18 - DNA, 23 bp, Artificial
  primer
gcagcgctctctataccaggtac

SEQ ID NO: 19 - DNA, 38 bp, Artificial
  primer
ttaatgtccatgttatgtctccgttatagctcatcgta

SEQ ID NO: 20 - DNA, 38 bp, Artificial
  primer
gtcgacggtcaccggcgccactaactcaacgctagtag

SEQ ID NO: 21 - DNA, 35 bp, Artificial
  primer
ttaattaagctagccagcaaagaaaaacaaacacg

SEQ ID NO: 22 - DNA, 27 bp, Artificial
  primer
ttaattaacaaatagttggtatagtcc

SEQ ID NO: 23 - DNA, 36 bp, Artificial
  primer
gacgatgccagcctgtcaccatggaaaactcctctc

SEQ ID NO: 24 - DNA, 46 bp, Artificial
  primer
cggatccctcgagctcagagcggccgcactagtcccggggagctcg

SEQ ID NO: 25 - DNA, 54 bp, Artificial
  primer
tcgacgagctccccgggactagtgcggccgctctgagctcgagggatccgacgt

(19) United States
(12) Patent Application Publication
(10) Pub. No.: US 2006/0135457 A1
(43) Pub. Date: Jun. 22, 2006

(54) Methods for constructing antibiotic resistance free vaccines

(76) Inventors: Yvonne Paterson, Philadelphia, PA (US); Thorsten Verch, North Wales, PA (US)

Correspondence Address: PEARL COHEN ZEDEK, LLP, 1500 BROADWAY 12TH FLOOR, NEW YORK, NY, 10036, US

(21) Appl. No.: 11/203,408

(22) Filed: Aug. 15, 2005

Related U.S. Application Data
(60) Provisional application No. 60/601,493, filed on Aug. 13, 2004.

(51) Int. Cl. A61K 48/00

(52) U.S. Cl. 514/44

(57) ABSTRACT

The present invention provides DNA vaccines comprising an antibiotic resistance gene-free plasmid, methods of generating same, and methods for treating a disease agent, comprising same.

### CROSS-REFERENCE TO RELATED APPLICATIONS

This application claims priority of U.S. Provisional Application Ser. No. 60/601,493, filed Aug. 13, 2004. This application is hereby incorporated in its entirety by reference herein.

### FIELD OF INVENTION

The present invention provides DNA vaccines comprising an antibiotic resistance gene-free plasmid, methods of generating same, and methods for treating a disease agent, comprising same.

### BACKGROUND OF THE INVENTION

Vaccines represent the most beneficial and cost effective public health measure currently known. However, as the understanding of neoplasias and infectious diseases grows, it has become apparent that traditional vaccine strategies my not be completely effective. Traditional vaccines have employed killed or attenuated pathogens or antigen subunits. The problem with this approach, especially with killed or subunit vaccines, is that the immune response in the vaccinated animal is primarily humoral in nature, and therefore not effective in combating intracellular organism or neoplasias that require cell mediated immunity for their destruction. In addition, attenuated or inactivated bacteria often only induce immunization for a short period of time and the immunity is limited to a humoral response. Further, traditional attenuated or inactivated bacterial vaccines do not elicit the cytotoxic T-lymphocyte (CTL) immune response necessary for the lysis of tumor cells and cells infected with intracellular pathogens.

Viral and bacterial vaccine vectors are often used to induce a CTL response. Viral vaccines are usually pathogenic viruses attenuated by serial passage in cell culture or viruses killed through heat or chemical inactivation. Killed viruses are incapable of infecting cells, and thus, like subunit vaccines, only elicit a humoral immune response. Attenuated viruses are capable of infecting cells, and can induce a CTL response in an individual. However, attenuated virus vaccines are not without drawbacks. First, attenuating a virus is often a process of trial and error. Bacterial vaccine vectors have also been employed to carry passenger antigens. For example Listeria monocytogenes (LM) vaccine vectors are adept at expressing a wide array of heterologous antigens and inducing a CTL response, as demonstrated by Paterson and Portnoy (U.S. Pat. No. 5,830,702). However, there are safety issue in using attenuated viruses and bacteria, especially in children, the elderly, and the immunocompromised.

A solution to the problems of traditional bacterial and viral vaccines exists in DNA vaccines. DNA vaccines are usually plasmids comprising a nucleic acid encoding an antigen, and elicit a strong humoral and cell-mediated immune response because the antigen is translated in the transfected cell, facilitating an MHC-mediated cellular response, and is expressed in the extracellular milieu, enabling a humoral response. Moreover, DNA vaccines can express a wide repertoire of proteins, including antigens, cytokines, and enzymes.

One drawback to DNA vaccines, however, is that in order to manufacture them in bacteria such as E. coli, it is necessary to include a drug resistance gene on the plasmid to select for retention of the plasmid by the bacteria during propagation. This requirement may cause concern over the spread of antibiotic resistance to microorganisms previously amenable to antibiotic therapy. Therefore, the presence of antibiotic resistance genes in a DNA vaccine is considered a liability from a safety perspective.

### BRIEF SUMMARY OF THE INVENTION

The present invention provides DNA vaccines comprising an antibiotic resistance gene-free plasmid, methods of generating same, and methods for treating a disease agent, comprising same.

In one embodiment, the present invention provides a DNA vaccine for generating an immune response against a protein antigen, the DNA vaccine comprising an antibiotic resistance gene-free plasmid, the antibiotic resistance gene-free plasmid comprising: (a) a first nucleic acid sequence encoding a polypeptide that comprises the protein antigen; and (b) a second nucleic acid sequence encoding a metabolic enzyme, wherein the antibiotic resistance gene-free plasmid is grown in an auxotrophic bacterial strain, whereby the metabolic enzyme complements a metabolic deficiency of the auxotrophic bacterial strain.

In another embodiment, the present invention provides a method of preparing a DNA vaccine for generating an immune response against a protein antigen, the method comprising (a) growing an auxotrophic bacterial strain containing a plasmid, wherein the plasmid comprises: (i) a first nucleic acid sequence encoding a polypeptide that comprises the protein antigen; and (ii) a second nucleic acid sequence encoding a metabolic enzyme, whereby the metabolic enzyme complements a metabolic deficiency of the auxotrophic bacterial strain; and wherein the plasmid does not contain an antibiotic resistance gene; and (b) isolating the plasmid DNA vaccine from the auxotrophic bacterial strain, thereby preparing a DNA vaccine for generating an immune response against a protein antigen.

### BRIEF DESCRIPTION OF THE DRAWINGS

FIG. 1 is a schematic map of E. coli-Listeria shuttle plasmids pGG55 (left side) and pTV3 (right side). CAT(−): E. coli chloramphenicol transferase; CAT(+): Listeria chloramphenicol transferase; Ori Lm: replication origin for Listeria; Ori Ec: p15 origin of replication for E. coli, prfA: Listeria pathogenicity regulating factor A, LLO: C-terminally truncated listeriolysin O including its promoter; E7: HPV E7; p60-dal; expression cassette of p60 promoter and Listerian alanine racemase gene. Selected restriction sites are also depicted.

FIG. 2: (A-D): Growth of MB2159 that has not been complemented with pTV3 showing that the strain requires alanine for growth and does not grow in the presence of chloramphenicol. E. coli strain MB2159 (alanine racemase negative), “empty.” Bacteria were plated on different media: A) LB−only MB2159 does not grow. B) LB+Ala−MB2159 grows. C) LB+Chloramphenicol−no bacteria grow D) LB+Chloramphenicol+Ala:—same as (c). (E-H): E. coli strain MB2159 containing pTV3 does not require D-alanine for growth and is chloramphenicol sensitive, showing that pTV3 provides the requirement for D-alanine but does not confer antibiotic resistance. E) LB−only complemented bacteria grow. F) LB+Ala−only MB2159 grows (with and without pTV3). G) LB+Chloramphenicol−no bacteria grow because the CAT gene is not present. H) LB+Chloramphenicol+Ala:—same as (G)

FIG. 3. Plasmid preparation of pTV3 from E. coli strain MB2159. Qiagen® midi-preparation of nucleic acids was following the manufacturer's protocol. Lanes from left to right: Lanes 1 and 7: Molecular Weight Marker, 100 Bp ladder (Invitrogen). Lane 2: pTV3, clone #15. Lane 3: pTV3, clone #16. Lane 4: pTV3C, clone #22. Lane 5: pTV3C, clone #24. Lane 6: pGG55 control.

### DETAILED DESCRIPTION OF THE INVENTION

The present invention provides DNA vaccines comprising an antibiotic resistance gene-free plasmid, methods of generating same, and methods for treating a disease agent, comprising same.

In one embodiment, the present invention provides a DNA vaccine for generating an immune response against a protein antigen, die DNA vaccine comprising an antibiotic resistance gene-free plasmid, the antibiotic resistance gene-free plasmid comprising: (a) a first nucleic acid sequence encoding a polypeptide that comprises the protein antigen; and (b) a second nucleic acid sequence encoding a metabolic enzyme, wherein the antibiotic resistance gene-free plasmid is grown in an auxotrophic bacterial strain, whereby the metabolic enzyme complements a metabolic deficiency of the auxotrophic bacterial strain.

In another embodiment, the present invention provides a DNA vaccine for conferring protection against a disease agent expressing a protein antigen, the DNA vaccine comprising an antibiotic resistance gene-free plasmid, the antibiotic resistance gene-free plasmid comprising: (a) a first nucleic acid sequence encoding a polypeptide that comprises the protein antigen; and (b) a second nucleic acid sequence encoding a metabolic enzyme, wherein the antibiotic resistance gene-free plasmid is grown in an auxotrophic bacterial strain, whereby the metabolic enzyme complements a metabolic deficiency of the auxotrophic bacterial strain.

In another embodiment, plasmids of methods and compositions of the present invention comprise a promoter/regulatory sequence operably linked to a gene encoding an antigen, amino acid metabolism gene, or a combination thereof. In another embodiment, the plasmid DNA vaccine of the present invention is replicated and propagated in a prokaryotic or eukaryotic host, such as a bacterium, and administered to an animal, e.g. a human. Therefore, in this embodiment, a plasmid DNA vaccine of the present invention comprises both eukaryotic and prokaryotic promoter/regulatory elements. Prokaryotic promoter/regulatory elements include, but are not limited to, T7, SP60, trp operon, tRNA promoters, lac operon, recA, lex A, and the like. Prokaryotic promoters are well known in the art, as are methods for their use. Each prokaryotic promoter/regulatory element represents a separate embodiment of the present invention.

In another embodiment, plasmids of methods and compositions of the present invention further comprise a eukaryotic promoter for expression of an antigen, or other protein from the plasmid DNA vaccine. A eukaryotic promoter useful in the present invention include constitutive, inducible, or tissue-specific promoters. Many promoter/regulatory sequences useful for driving constitutive expression of a gene are available in the art and include, but are not limited to, for example, the cytomegalovirus immediate early promoter enhancer sequence, the SV40 early promoter, the immunoglobulin promoter, as well as the Rous sarcoma virus promoter, and the like. Moreover, inducible and tissue specific expression of an antigen, or enzyme can be accomplished by placing the nucleic acid encoding such a protein under the control of an inducible or tissue specific promoter/regulatory sequence. Examples of tissue specific or inducible promoter/regulatory sequences which are useful for his purpose include, but are not limited to the MMTV LTR inducible promoter, and the SV40 late enhancer/promoter. In addition, promoters which are well known in the art which are induced in response to inducing agents such as metals, glucocorticoids, tetracycline, hormones, and the like, are also contemplated in the invention. Thus, it will be appreciated that the invention includes the use of any promoter/regulatory sequence known in the art that is capable of driving expression of the desired protein operably linked thereto.

In another embodiment, plasmids of methods and compositions of the present invention have been specifically optimized for polynucleotide vaccinations. Elements include a transcriptional promoter, immunogenic epitopes, and additional cistrons encoding immunoenhancing or immunomodulatory genes, with their own promoters, transcriptional terminator, bacterial origin of replication and antibiotic resistance gene, as described herein. In another embodiment, the vector contains internal ribosome entry sites (IRES) for the expression of polycistronic mRNA. Those skilled in the art will appreciate that RNA which has been transcribed in vitro to produce multi-cistronic mRNAs encoded by the DNA counterparts is within the scope of this invention. For this purpose, it is desirable to use as the transcriptional promoter such powerful RNA polymerase promoters as the T7 or SP6 promoters, and performing in vitro run-on transcription with a linearized DNA template. These methods are well known in the art, and are described in detail in, for example, Lowrie and Whalen (eds.) (DNA Vaccines: Methods and Protocols (Methods in Molecular Medicine, No. 29, 2000, Humana Press, Totowa N.J.).

In one embodiment, the auxotrophic bacterial strain of methods and compositions of the present invention is an auxotrophic E. coli strain. In another embodiment, the auxotrophic bacterial strain is any other bacterial strain known in the art that has utility in growing a plasmid. Each possibility represents a separate embodiment of the present invention.

In another embodiment, a DNA vaccine of methods and compositions of the present invention further comprises an adjuvant. In another embodiment, the DNA vaccine further comprises a cytokine-encoding nucleotide molecule. In another embodiment, the DNA vaccine further comprises a pharmaceutically acceptable carrier. Each possibility represents a separate embodiment of the present invention.

In another embodiment, the present invention provides a method for treating a disease agent, wherein the disease agent expresses a protein antigen of the present invention, comprising administering a DNA vaccine of the present invention.

In one embodiment, the disease agent is a pathogen. In another embodiment, the disease agent is a cancer cell. In another embodiment, the disease agent is a neoplastic cell. In another embodiment, the disease agent is any other type of disease agent known in the art. Each possibility represents a separate embodiment of the present invention.

In another embodiment, the present invention provides a method of preparing a DNA vaccine for generating an immune response against a protein antigen, the method comprising (a) growing an auxotrophic bacterial strain containing a plasmid, wherein the plasmid comprises: (i) a first nucleic acid sequence encoding a polypeptide that comprises the protein antigen; and (ii) a second nucleic acid sequence encoding a metabolic enzyme, whereby the metabolic enzyme complements a metabolic deficiency of the auxotrophic bacterial strain; and wherein the plasmid does not contain an antibiotic resistance gene; and (b) isolating the plasmid DNA vaccine from the auxotrophic bacterial strain, thereby preparing a DNA vaccine for generating an immune response against a protein antigen.

In another embodiment, a method of the present invention further comprises contacting an auxotrophic bacterial strain with a plasmid of the present invention, whereby the auxotrophic bacterial strain takes up the plasmid. In another embodiment, a method of the present invention further comprises transforming an auxotrophic bacterial strain with a plasmid of the present invention. In another embodiment, methods of the present invention utilize bacterial strain that have already been transfected. Each possibility represents a separate embodiment of the present invention.

“Transforming,” in one embodiment, is used identically with the term “transfecting,” and refers to engineering a bacterial cell to take up a plasmid or other heterologous DNA molecule. In another embodiment, “transforming” refers to engineering a bacterial cell to express a gene of a plasmid or other heterologous DNA molecule. Each possibility represents a separate embodiment of the present invention.

In another embodiment, the plasmid of methods and compositions of the present invention further comprises a transcription factor. In another embodiment, the transcription factor is lacking in the auxotrophic bacterial strain or in the bacteria chromosome of a bacterial strain of the present invention. In various embodiments, the transcription factor is any other transcription factor known in the art.

In one embodiment, the metabolic gene, transcription factor, etc. is lacking in a chromosome of the bacterial strain. In another embodiment, the metabolic gene, transcription factor, etc. is lacking in all the chromosomes of the bacterial strain. In another embodiment, the metabolic gene, transcription factor, etc. is lacking in the genome of the bacterial strain.

In one embodiment, the transcription factor is mutated in the chromosome. In another embodiment, the transcription factor is deleted from the chromosome. Each possibility represents a separate embodiment of the present invention.

In another embodiment, the plasmid of methods and compositions of the present invention does not confer antibiotic resistance to the bacterial vaccine strain. In another embodiment, the plasmid does not contain an antibiotic resistance gene. Each possibility represents a separate embodiment of the present invention.

In another embodiment of methods and compositions of the present invention, a polypeptide encoded by a nucleic acid sequence of methods and compositions of the present invention is a fusion protein comprising the heterologous antigen and an additional polypeptide.

In another embodiment, a fusion protein of the present invention comprises, inter alia, an LM non-hemolytic LLO protein (Examples herein). The non-hemolytic LLO protein comprises, in one embodiment, about the first 400 to 441 amino acids of the 529 amino acid full-length LLO protein, the sequence of which is described in, for example, Mengaud et al, (1988, Infect. Immun. 56:766-772, GenBank Acc. No. P13128). The construction of a fusion protein comprising an antigen and a non-hemolytic LLO protein is described elsewhere herein, and in, for example, Gunn et al, (2001, J. Immunology 167: 6471-6479).

In another embodiment, a fusion protein of methods and compositions of the present invention comprises a PEST sequence, either from an LLO protein or from another organism e.g. a prokaryotic organism.

In another embodiment, a fusion protein of methods and compositions of the present invention comprises an Act A sequence from a Listeria organism. The construction and use of a fusion protein comprising a PEST sequence or an ActA sequence proceeds as described herein and in U.S. Pat. No. 6,767,542, International Publication No. WO 01172329 and U.S. application Ser. No. 10/835,662 of Paterson et al. ActA proteins and fragments thereof augment antigen presentation and immunity in a similar fashion to LLO.

In another embodiment, the additional polypeptide is any other polypeptide known in the art. Each of the above additional polypeptides represents a separate embodiment of the present invention.

Fusion proteins comprising an antigen may be prepared by any suitable method, including, for example, cloning and restriction of appropriate sequences or direct chemical synthesis by methods discussed below. Alternatively, subsequences may be cloned and the appropriate subsequences cleaved using appropriate restriction enzymes. The fragments may then be ligated to produce the desired DNA sequence. In one embodiment, DNA encoding the antigen can be produced using DNA amplification methods, for example polymerase chain reaction (PCR). First, the segments of the native DNA on either side of the new terminus are amplified separately. The 5′ end of the one amplified sequence encodes the peptide linker, while the 3′ end of the other amplified sequence also encodes the peptide linker. Since the 5′ end of the first fragment is complementary to the 3′ end of the second fragment, the two fragments (after partial purification, e.g. on LMP agarose) can be used as an overlapping template in a third PCR reaction. The amplified sequence will contain codons, the segment on the carboxy side of the opening site (now forming the amino sequence), the linker, and the sequence on the amino side of the opening site (now forming the carboxyl sequence). The antigen is ligated into a plasmid. Each method represents a separate embodiment of the present invention.

In another embodiment, the first nucleic acid sequence of methods and compositions of the present invention is operably linked to a promoter/regulatory sequence. In one embodiment, the promoter/regulatory sequence is p60, which the present invention shows is operational in E. coli. In another embodiment, the second nucleic acid sequence is operably linked to a promoter/regulatory sequence. In another embodiment, each of the nucleic acid sequences is operably linked to a promoter/regulatory sequence. Each possibility represents a separate embodiment of the present invention.

In another embodiment, the a promoter/regulatory sequence of the second nucleic acid sequence functions in E. coli, thereby enabling stable maintenance of the plasmid in the E. coli strain. In another embodiment, the second nucleic acid sequence is expressed in an E. coli strain upon transfecting the E. coli strain with a plasmid of the present invention, thereby enabling stable maintenance of the plasmid in the E. coli strain.

In another embodiment of methods and compositions of the present invention, a metabolic enzyme encoded by a nucleic acid sequence thereof is an amino acid metabolism enzyme. In another embodiment, the metabolic enzyme is an alanine racemase enzyme. In another embodiment, the metabolic enzyme is a D-amino acid transferase enzyme. The LM alanine racemase and D-amino acid transferase genes were cloned and isolated from LM as described in Thompson et al (Infec Immun 66: 3552-3561, 1998). In another embodiment, the metabolic gene is one or more of: AroA, B and E genes, e.g for Listeria. In another embodiment, the metabolic gene is one or more of: AroA, AroB, AroC, AroD, AroE, AroG, AroK and TrpS, e.g. for E. coli.

In another embodiment, an alanine racemase gene utilized in the present invention has the sequence set forth in GenBank Accession Number AF038438. In another embodiment, the alanine racemase gene is any another alanine racemase gene known in the art. Each possibility represents a separate embodiment of the present invention.

In another embodiment, a D-amino acid transferase gene utilized in the present invention has the sequence set forth in GenBank Accession Number AF038439. In another embodiment, the D-amino acid transferase gene is any another D-amino acid transferase gene known in the art. Each possibility represents a separate embodiment of the present invention.

Bacteria auxotrophic for D-alanine synthesis are well known in the art, and are described in, for example, Strych et al, (J. Bacteriol. 184:4321-4325, 2002). Each auxotrophic bacterial strain represents a separate embodiment of the present invention.

In various embodiments, the antigen of methods and compositions of the present invention is derived from a tumor or an infectious organism, including, but not limited to, fungal pathogens, bacteria, parasites, helminths, viruses, and the like. An antigen of the present invention includes but is not limited to, tetanus toxoid, hemagglutinin molecules from influenza virus, diphtheria toxoid, HIV gp120, HIV gag protein, IgA protease, insulin peptide B, Spongospora subterranea antigen, vibriose antigens, Salmonella antigens, pneumococcus antigens, respiratory syncytial virus antigens, Haemophilus influenza outer membrane proteins, Helicobacter pylori urease, Neisseria meningitidis pilins, N. gonorrhoeae pilins, the melanoma-associated antigens (TRP-2, MAGE-1, MAGE-3, gp-100, tyrosinase, MART-1, HSP-70, beta-HCG), human papilloma virus antigens E1, E2, E6 and E7 from type HPV-16, -18, -31, -33, -35 or 45 human papilloma viruses, the tumor antigens Her2/neu (e.g. GenBank Accession No. MI 1730), NY-ESO1 (e.g. GenBank Accession No. NM_{—}001327) CEA, the ras protein, mutated or otherwise, the p53 protein, mutated or otherwise, Mucl, pSA, the antigens well known in the art from the following diseases; cholera, diphtheria, Haemophilus, hepatitis A, hepatitis B, influenza, measles, meningitis, mumps, pertussis, small pox, pneumococcal pneumonia, polio, rabies, rubella, tetanus, tuberculosis, typhoid, Varicella-zoster, whooping cough, yellow fever, the immunogens and antigens from Addison's disease, allergies, anaphylaxis, Bruton's syndrome, cancer, including solid and blood borne tumors, eczema, Hashimoto's thyroiditis, polymyositis, dermatomyositis, type 1 diabetes mellitus, acquired immune deficiency syndrome, transplant rejection, such as kidney, heart, pancreas, lung, bone, and liver transplants, Graves' disease, polyendocrine autoimmune disease, hepatitis, microscopic polyarteritis, polyarteritis nodosa, pemphigus, primary biliary cirrhosis, pernicious anemia, coeliac disease, antibody-mediated nephritis, glomerulonephritis, rheumatic diseases, systemic lupus erthematosus, rheumatoid arthritis, seronegative spondylarthritides, rhinitis, sjogren's syndrome, systemic sclerosis, sclerosing cholangitis, Wegener's granulomatosis, dermatitis herpetiformis, psoriasis, vitiligo, multiple sclerosis, encephalomyelitis, Guillain-Barre syndrome, myasthenia gravis, Lambert-Eaton syndrome, sclera, episclera, uveitis, chronic mucocutaneous candidiasis, urticaria, transient hypogammaglobulinemia of infancy, myeloma, X-linked hyper IgM syndrome, Wiskott-Aldrich syndrome, ataxia telangiectasia, autoimmune hemolytic anemia, autoimmune thrombocytopenia, autoimmune neutropenia, Waldenstrom's macroglobulinemia, amyloidosis, chronic lymphocytic leukemia, non-Hodgkin's lymphoma, malarial circumsporozite protein, microbial antigens, viral antigens, autoantigens, and listeriosis.

In another embodiment, tumor antigens utilized in the present invention include, but are not limited to, any of the various MAGEs (Melanoma-Associated Antigen E), including MAGE 1 (e.g., GenBank Accession No. M77481), MAGE 2 (e.g., Genbank Accession No. U03735), MAGE 3, MAGE 4, etc.; any of the various tyrosinases; mutant ras; mutant p53 (e.g., GenBank Accession No. X54156 and AA494311); and p97 melanoma antigen (e.g., GenBank Accession No. M12154). Other tumor-specific antigens include the Ras peptide and p53 peptide associated with advanced cancers, the HPV 16/18 and E6/E7 antigens associated with cervical cancers, MUC1-KLH antigen associated with breast carcinoma (e.g., GenBank Accession No. J03651), CEA (carcinoembryonic antigen) associated with colorectal cancer (e.g., GenBank Accession No. X98311), gp1OO (e.g., GenBank Accession No. S73003) or MART1 antigens associated with melanoma, and the PSA antigen associated with prostate cancer (e.g., GenBank Accession No. X14810). The p53 gene sequence is known (See e.g., Harris et al. (1986) Mol. Cell. Biol., 6:4650-4656) and is deposited with GenBank under Accession No. M14694. Her-2/Neu (e.g. GenBank Accession Nos. M16789.1, M16790.1, M16791.1, M16792.1), NY-ESO-1 (e.g. GenBank Accession No. U87459), hTERT (aka telomerase) (GenBank Accession. Nos. NM003219 (variant 1), NM198255 (variant 2), NM 198253 (variant 3), and NM 198254 (variant 4), proteinase 3 (e.g. GenBank Accession Nos. M29142, M75154, M96839, X55668, NM 00277, M96628 and X56606) HPV E6 and E7 (e.g. GenBank Accession No. NC 001526) and WT-1 (e.g. GenBank Accession Nos. NM000378 (variant A), NM024424 (variant B), NM 024425 (variant C), and NM024426 (variant D)). Thus, the present invention can be used as immunotherapeutics for cancers including, but not limited to, cervical, breast, colorectal, prostate, lung cancers, and for melanomas.

In other embodiments, the antigen is an antigen from one of the following infectious diseases; measles, mumps, rubella, poliomyelitis, hepatitis A, B (e.g., GenBank Accession No. E02707), and C (e.g., GenBank Accession No. E06890), as well as other hepatitis viruses, influenza, adenovirus (e g., types 4 and 7), rabies (e.g., GenBank Accession No. M34678), yellow fever, Japanese encephalitis (e.g., GenBank Accession No. E07883), dengue (e.g., GenBank Accession No. M24444), hantavirus, and HIV (e.g., GenBank Accession No. U18552). Bacterial and parasitic antigens will be derived from known causative agents responsible for diseases including, but not limited to, diphtheria, pertussis (e.g., GenBank Accession No. M35274), tetanus (e.g., GenBank Accession No. M64353), tuberculosis, bacterial and fungal pneumonias (e.g., Haemophilus influenzae, Pneumocystis carinii, etc.), cholera, typhoid, plague, shigellosis, salmonellosis (e.g., GenBank Accession No. L03833), Legionnaire's Disease, Lyme disease (e.g., GenBank Accession No. U59487), malaria (e.g., GenBank Accession No. X53832), hookworm, onchocerciasis (e.g., GenBank Accession No. to M27807), schistosomiasis (e.g., GenBank Accession No. L08198), trypanosomiasis, leshmaniasis, giardiasis (e.g., GenBank Accession No. M33641), amoebiasis, filariasis (e.g., GenBank Accession No. J03266), borreliosis, and trichinosis.

Each of the above antigens represents a separate embodiment of the present invention.

In one embodiment, an advantage of DNA vaccines of the present invention is that this system can be used for plasmids of any copy number. In another embodiment, DNA vaccines of the present invention are not restricted to high-copy-number plasmids, as in the alternative repressor titration system. In another embodiment, the advantage is lack of necessity to introduce potentially toxic amounts of D-alanine. Each possibility represents a separate embodiment of the present invention.

In another embodiment, the present invention provides a kit comprising a DNA vaccine of the present invention, a pharmaceutically-acceptable carrier, an applicator, and an instructional material for use thereof.

In another embodiment, the present invention provides a kit comprising a DNA vaccine of the present invention, an applicator, and an instructional material for use thereof.

“Alanine racemase” refers, in one embodiment, to an enzyme that converts the L-isomer of the amino acid alanine into its D-isomer. In another embodiment, such enzymes are known by the EC number 5.1.1.1.

“Amino acid metabolism enzyme” refers, in one embodiment, to a peptide or protein that has a functional role in converting an amino acid from one form to another, such as, but not limited to, altering the stereochemistry of the amino acid, hydrolyzing or adding groups to an amino acid, cleaving amino acids, and the like. Each possibility represents a separate embodiment of the present invention.

The term “auxotrophic bacteria” refers, in one embodiment, to a bacteria strain that is not capable of growing or replicating without supplementation of a factor that will permit such growth or replication. Each factor represents a separate embodiment of the present invention.

“Fusion protein” refers, in one embodiment, to a protein that comprises two or more proteins linked together. In one embodiment, the proteins are linked by peptide bonds. In another embodiment, the proteins are linked by other chemical bonds. In another embodiment, the proteins are linked by with one or more amino acids between the two or more proteins, which may be referred to as a spacer. Each possibility represents a separate embodiment of the present invention.

“Homologous” refers, in one embodiment, to the subunit sequence similarity between two polymeric molecules, e.g., between two nucleic acid molecules, e.g., two DNA molecules or two RNA molecules, or between two polypeptide molecules. When a subunit position in both of the two molecules is occupied by the same monomeric subunit, e.g., if a position in each of two DNA molecules is occupied by adenine, then they are homologous at that position. In another embodiment, the homology between two sequences is a direct function of the number of matching or homologous positions, e.g., if half (e.g., five positions in a polymer ten subunits in length) of the positions in two compound sequences are homologous then the two sequences are 50% homologous, if 90% of the positions, e.g., 9 of 10, are matched or homologous, the two sequences share 90% homology. By way of example, the DNA sequences 3′ATTGCC5′ and 3′TATGGC share 50% homology. In another embodiment, “homology” is used synonymously with “identity.” In another embodiment, when the terms “homology” or “identity” are used herein to refer to the nucleic acids and proteins, it should be construed to be applied to homology or identity at both the nucleic acid and the ammo acid sequence levels.

In another embodiment, the terms “gene” and “recombinant gene” refer to nucleic acid molecules comprising an open reading frame encoding a polypeptide of the invention. Such natural allelic variations can typically result in 1-5% variance in the nucleotide sequence of a given gene. Alternative alleles can be identified by sequencing the gene of interest in a number of different individuals or organisms. This can be readily carried out by using hybridization probes to identify the same genetic locus in a variety of individuals or organisms. Any and all such nucleotide variations and resulting amino acid polymorphisms or variations that are the result of natural allelic variation and that do not alter the functional activity are intended to be within the scope of the invention.

Describing two polynucleotides as “operably linked” means, in one embodiment, that a single-stranded or double-stranded nucleic acid moiety comprises the two polynucleotides arranged within the nucleic acid moiety in such a manner that at least one of the two polynucleotides is able to exert a physiological effect by which it is characterized upon the other. By way of example, a promoter operably linked to the coding region of a gene is able to promote transcription of the coding region.

“Promoter/regulatory sequence” refers, in one embodiment, to a nucleic acid sequence which is required for, or enhances, expression of a gene product operably linked to the promoter/regulatory sequence. In another embodiment, this sequence is the core promoter sequence. In another embodiment, this sequence also includes an enhancer sequence and other regulatory elements that are required for expression of the gene product.

The skilled artisan, when equipped with the present disclosure and the methods herein, will readily understand that different transcriptional promoters, terminators, carrier vectors or specific gene sequences (e.g. those in commercially available cloning vectors) may be used successfully in DNA vaccines of methods and compositions of the present invention. As is contemplated in the present invention, these functionalities are provided in, for example, the commercially available vectors known as the pUC series. In another embodiment, non-essential DNA sequences (e.g. antibiotic resistance genes) are removed.

In another embodiment, a commercially available plasmid is used in the present invention. Such plasmids are available from a variety of sources, for example, Invitrogen (La Jolla, Calif.), Stratagene (La Jolla, Calif.), Clontech (Palo Alto, Calif.), or can be constructed using methods well known in the art. Another embodiment is a plasmid such as pCR2.1 (Invitrogen, La Jolla, Calif.), which is a prokaryotic expression vector with a prokaryotic origin of replication and promoter/regulatory elements to facilitate expression in a prokaryotic organism. In another embodiment, extraneous nucleotide sequences are removed to decrease the size of the plasmid and increase the size of the cassette that may be placed therein.

In another embodiment, antibiotic resistance genes are removed from a plasmid DNA vaccine using any number of methods well known in the art, including, but not limited to, restriction endonuclease digestion, removal of a promoter driving expression of the antibiotic resistance gene, partial restriction digestion of an antibiotic resistance gene, point mutagenesis, insertional mutagenesis, and the like.

Commercially available plasmids are available from a variety of sources, for example, Invitrogen (La Jolla, Calif.). In another embodiment, a commercially available plasmid such as pcDNA3.1(+), which is a eukaryotic expression vector with an prokaryotic origin of replication to allow propagation according to the methods of the present invention is utilized. In another embodiment, extraneous nucleotide sequences are removed to decrease the size of the plasmid DNA vaccine and increase the size of the cassette that may be placed therein. In another embodiment, sequences with homology to human or other animal genomic sequences are removed to prevent homologous recombination and insertional mutagenesis in the human genome. In another embodiment the commercially available plasmid is then used for propagation of a plasmid DNA vaccine in an auxotrophic bacterium.

Such methods are well known in the art, and are described in, for example, Sambrook et al. (1989, Molecular Cloning: A Laboratory Manual, Cold Spring Harbor Laboratory Press, New York) and Ausubei et al. (1997, Current Protocols in Molecular Biology, Green & Wiley, New York).

Antibiotic resistance genes are used in the conventional selection and cloning processes commonly employed in molecular biology and vaccine preparation. Antibiotic resistance genes contemplated in the present invention include, but are not limited to, gene products that confer resistance to ampicillin, penicillin, methicillin, streptomycin, erythromycin, kanamycin, tetracycline, cloramphenicol (CAT), neomycin, hygromycin, gentamicin and others well known in the art. Each gene represents a separate embodiment of the present invention.

Methods for removing or inactivating an antibiotic resistance gene in a plasmid are well known in the art. In one embodiment, the available restriction sites in an antibiotic resistance gene in a plasmid DNA vaccine are identified using the program NEB cutter v 1.0, publicly available from New England Biolabs (Beverly, Mass.). The antibiotic resistance gene is then cut with the restriction enzymes specific for the restriction sites identified, thus removing the nucleic acid between the two restriction sites. After restriction with appropriate enzymes, the plasmid DNA vaccine is re-ligated or blunt end re-ligated, thus inactivating the antibiotic resistance gene. In another embodiment, this method is performed multiple times if the plasmid DNA vaccine comprises more than one antibiotic resistance gene. In another embodiment, similar methods are applied to inactivate the promoter for an antibiotic resistance gene. Thus, the promoter/regulatory element of an antibiotic resistance gene is restriction digested, thereby limiting, inhibiting, or ceasing expression of the antibiotic resistance gene. The sequences of antibiotic resistance genes are well known in the art.

Methods for transforming bacteria are well known in the art, and include calcium-chloride competent cell-based methods, electroporation methods, bacteriophage-mediated transduction, chemical, and physical transformation techniques (de Boer et al, 1989, Cell 56:641-649; Miller et al, 1995, FASEB J., 9:190-199; Sambrook et al. 1989, Molecular Cloning: A Laboratory Manual, Cold Spring Harbor Laboratory, New York; Ausubel et al., 1997, Current Protocols in Molecular Biology, John Wiley & Sons, New York; Gerhardt et al., eds., 1994, Methods for General and Molecular Bacteriology, American Society for Microbiology, Washington, D.C.; Miller, 1992, A Short Course in Bacterial Genetics, Cold Spring Harbor Laboratory Press, Cold Spring Harbor, N.Y.) In one embodiment, the auxotrophic bacterial strain of the present invention is transformed by electroporation. Each method represents a separate embodiment of the present invention.

Plasmids and other expression vectors useful in the present invention are described elsewhere herein, and can include such features as a promoter/regulatory sequence, an origin of replication for gram negative and gram positive bacteria, an isolated nucleic acid encoding a fusion protein and an isolated nucleic acid encoding an amino acid metabolism gene. Further, an isolated nucleic acid encoding a fusion protein and an amino acid metabolism gene will have a promoter suitable for driving expression of such an isolated nucleic acid. Promoters useful for driving expression in a bacterial system are well known in the art, and include bacteriophage lambda, the bla promoter of the beta-lactamase gene of pBR322, and the CAT promoter of the chloramphenicol acetyl transferase gene of pBR325. Further examples of prokaryotic promoters include the major right and left promoters of bacteriophage lambda (P_{L} and P_{R}), the trp, recA, lacZ, lad, and gal promoters of E. coli, the alpha-amylase (Ulmanen et al, 1985. J. Bacteriol. 162:176-182) and the S28-specific promoters of B. subtilis (Gilman et al, 1984 Gene 32:11-20), the promoters of the bacteriophages of Bacillus (Gryczan, 1982, In: The Molecular Biology of the Bacilli, Academic Press, Inc., New York), and Streptomyces promoters (Ward et al, 1986, Mol. Gen. Genet. 203:468-478). Additional prokaryotic promoters contemplated in the present invention are reviewed in, for example, Glick (1987, J. Ind. Microbiol. 1:277-282); Cenatiempo, (1986, Biochimie, 68:505-516); and Gottesman, (1984, Ann. Rev. Genet. 18:415-442).

The gene expressed on a plasmid of a DNA vaccine of methods and compositions of the present invention comprises, in one embodiment, an isolated nucleic acid encoding a protein that complements the auxotrophic mutant. In another embodiment, if the auxotrophic bacteria is deficient in a gene encoding a vitamin synthesis gene (e.g. pantothenic acid) necessary for bacterial growth, the plasmid DNA vaccine comprises a gene encoding a protein for pantothenic acid synthesis. Thus, the auxotrophic bacteria, when expressing the gene on the plasmid, can grow in the absence of pantothenic acid, whereas an auxotrophic bacteria not expressing the gene on the plasmid cannot grow in the absence of pantothenic acid.

In another embodiment, the plasmid comprises a gene encoding an amino acid metabolism enzyme. Such enzymes metabolize amino acids such that they can be used for bacterial growth and replication processes, such as cell wall synthesis, protein synthesis, fatty acid metabolism, and the like. In another embodiment, an auxotrophic bacteria is deficient in the amino acid metabolism enzymes for D-glutamic acid, a cell wall component. D-glutamic acid synthesis is controlled by the D-amino acid transferase gene, which is involved in the conversion of D-glu+pyr to alpha-ketoglutarate+D-ala, and the reverse reaction. D-glutamic acid synthesis is also controlled by the dga gene, and an auxotrophic mutant for D-glutamic acid synthesis will not grow in the absence of D-glutamic acid (Pucci et al, 1995, J Bacteriol. 177: 336-342). A further example includes a gene involved in the synthesis of diaminopimelic acid. Such synthesis genes encode beta-semialdehyde dehydrogenase, and when inactivated, renders a mutant auxotrophic for this synthesis pathway (Sizemore et al, 1995, Science 270: 299-302).

The recombinant proteins of the present invention are synthesized, in another embodiment, using recombinant DNA methodology. This involves, in one embodiment, creating a DNA sequence that encodes the fusion protein, placing the DNA in an expression cassette, such as the plasmid of the present invention, under the control of a particular promoter/regulatory element, and expressing the protein. DNA encoding the fusion protein (e.g. non-hemolytic LLO/antigen) of the present invention may be prepared by any suitable method, including, for example, cloning and restriction of appropriate sequences or direct chemical synthesis by methods such as the phosphotriester method of Narang et al. (1979, Meth. Enzymol. 68: 90-99); the phosphodiester method of Brown et al. (1979, Meth. Enzymol 68: 109-151); the diethylphosphoramidite method of Beaucage et al. (1981, Tetra. Lett., 22: 1859-1862); and the solid support method of U.S. Pat. No. 4,458,066.

In another embodiment, chemical synthesis is used to produce a single stranded oligonucleotide. This single stranded oligonucleotide is converted, in various embodiments, into double stranded DNA by hybridization with a complementary sequence, or by polymerization with a DNA polymerase using the single strand as a template. One of skill in the art would recognize that while chemical synthesis of DNA is limited to sequences of about 100 bases, longer sequences may be obtained by the ligation of shorter sequences. In another embodiment, subsequences are cloned and the appropriate subsequences cleaved using appropriate restriction enzymes. The fragments are then be ligated to produce the desired DNA sequence.

In another embodiment, DNA encoding the fusion protein or the recombinant protein of the present invention may be cloned using DNA amplification methods such as polymerase chain reaction (PCR). Thus, the gene for non-hemolytic LLO is PCR amplified, using a sense primer comprising a suitable restriction site and an antisense primer comprising another restriction site, e.g. a non-identical restriction site to facilitate cloning. The same is repeated for the isolated nucleic acid encoding an antigen. Ligation of the non-hemolytic LLO and antigen sequences and insertion into a plasmid or vector produces a vector encoding non-hemolytic LLO joined to a terminus of the antigen. The two molecules are joined either directly or by a short spacer introduced by the restriction site.

In another embodiment, the molecules are separated by a peptide spacer consisting of one or more amino acids, generally the spacer will have no specific biological activity other than to join the proteins or to preserve some minimum distance or other spatial relationship between them. However, the constituent amino acids of the spacer may be selected to influence some property of the molecule such as the folding, net charge, or hydrophobicity. In another embodiment, the plasmid further comprises additional promoter regulatory elements, as well as a ribosome binding site and a transcription termination signal. In another embodiment, the control sequences include a promoter and an enhancer derived from e.g. immunoglobulin genes, SV40, cytomegalovirus, etc., and a polyadenylation sequence. In another embodiment, the control sequences include splice donor and acceptor sequences.

In another embodiment, the antigen of methods and compositions of the present invention

The antigens of these and other diseases are well known in the art, and the skilled artisan, when equipped with the present disclosure and the methods and techniques described herein will readily be able to construct a fusion protein comprising a non-hemolytic LLO protein and an antigen for use in the present invention. In another embodiment, in order to select for an auxotrophic bacteria comprising the plasmid, transformed auxotrophic bacteria are grown on a media that will select for expression of the amino acid metabolism gene. For example, in one embodiment, a bacteria auxotrophic for D-glutamic acid synthesis is transformed with a plasmid comprising a gene for D-glutamic acid synthesis, and the auxotrophic bacteria will grow in the absence of D-glutamic acid, whereas auxotrophic bacteria that have not been transformed with the plasmid, or are not expressing the plasmid encoding a protein for D-glutamic acid synthesis, will not grow. In another embodiment, a bacteria auxotrophic for D-alanine synthesis will grow in the absence of D-alanine when transformed and expressing the plasmid of the present invention if the plasmid comprises an isolated nucleic acid encoding an amino acid metabolism enzyme for D-alanine synthesis. Such methods for making appropriate media comprising or lacking necessary growth factors, supplements, amino acids, vitamins, antibiotics, and the like are well known in the art, and are available commercially (Becton-Dickinson, Franklin Lakes, N.J.). Each method represents a separate embodiment of the present invention.

In another embodiment, once the auxotrophic bacteria comprising the plasmid of the present invention have been selected on appropriate media, the bacteria are propagated in the presence of a selective pressure. Such propagation comprises growing the bacteria in media without the auxotrophic factor. The presence of the plasmid expressing an amino acid metabolism enzyme in the auxotrophic bacteria ensures that the plasmid will replicate along with the bacteria, thus continually selecting for bacteria harboring the plasmid. The skilled artisan, when equipped with the present disclosure and methods herein will be readily able to scale-up the production of the DNA vaccine by adjusting the volume of the media in which the auxotrophic bacteria comprising the plasmid are growing.

### EXPERIMENTAL DETAILS SECTION

### Example 1

### A Plasmid Containing an Amino Acid Metabolism Enzyme Instead of an Antibiotic Resistance Gene is Retained in Auxotrophic Bacteria both in Vitro and in Vivo

### Material and Experimental Methods

Transformation and Selection

E. coli strain MB2159 was used for transformations, using standard protocols. Bacterial cells were prepared for electroporation by washing with H₂O.

Bacterial Culture and In Vivo Passaging of E. coli

E. coli were cultured following standard methods. For growth kinetics determinations, bacteria were grown for 16 hours in 10 ml of LB+antibiotics. The OD_{600 nm} was measured and culture densities were normalized between the strains. The culture was diluted 1:50 into LB+suitable antibiotics and D-alanine if applicable.

### Construction of Antibiotic Resistance Factor Free Plasmid pTV3

The starting point for subcloning of pGG55 was the plasmid pDP1659. pDP1659 was generated by PCR (polymerase chain reaction)-amplifying from LM genomic DNA the DNA fragment encoding first 420 amino acids of LLO together with the upstream regulatory sequences and promoter, then ligating the fragment into pUC19. The DNA fragment encoding the NP antigen was PCR amplified, using plasmid pAPR501, provided by Dr. Peter Palese, as a template, and ligated into pUC19 as an in-frame translational fusion downstream of the LLO fragment. The fusion protein was then subcloned into pAM401, a shuttle vector able to replicate in both gram-negative and gram-positive bacteria (Wirth R et al. Highly efficient protoplast transformation system for Streptococcus faecalis and a new Escherichia coli-S. faecalis shuttle vector. J Bacteriol 165(3): 831-6, 1986). The hly promoter and gene fragment were generated using primers 5′-GGGGGCTAGCCCTCCTTTGATTAGTATATTC-3′ (SEQ ID NO: 3) and 5′-CTCCCTCGAGATCATAATTTACTTCATC-3′ (SEQ ID NO: 4).

Next, plasmid pDP2028 was constructed by cloning the prfA gene into the SalI site of pDP1659. The prfA gene was PCR amplified using the following primers:

| | |
|---|---|
| 5′GACTACAAGGACGATGACCGACAAGTGATAACCCG | (SEQ ID NO:5) |
| GGATCTAAATAAATCCGTTT-3′ | |
| and | |
| 5′-CCCGTCGACCAGCTCTTCTTGGTGAAG-3′. | (SEQ ID NO:6) |

pGG34 was next created from pDP2028 and pGG49. pGG49 contains an insert that consists of the hly-promoter, a gene encoding an N-terminal LLO fragment fused with HIV gp70, and the Listeria prfA gene. pGG49 was digested with NheI and SalI to remove the insert, which was ligated into XbaI and SalI-digested pDP2028 to yield pGG34.

pGG55 was then generated from pGG34 as follows: The human papilloma virus E7 gene was amplified by PCR using the primers 5′-GGCTCGAGCATGGAGATACACC-3′ (SEQ ID NO: 1) and 5′-GGGGACTAGTTTATGGTTTCTGAGAACA-3′ (SEQ ID NO: 2), digested with XhoI and SpeI (New England Biolabs, Beverly, Mass.), and ligated into similarly digested pGG34, thereby fusing the E7 gene to the hly gene that is located upstream of XhoI. The resulting plasmid is pGG55 which contains a multi-gene cassette of hly, E7 antigen and prfA. The hly promoter drives the expression of the first 441 amino acids of the hly gene product, LLO, which is joined by the XhoI site to the E7 gene. By deleting the hemolytic C-terminus of LLO, the hemolytic activity of the fusion protein is neutralized. The pluripotent transcription factor, prfA, is also included on pGG-55 with its expression driven by its natural promoter.

Construction of p60-Alanine Racemase Cassette

Next, a fusion of a truncated p60 promoter to the alanine racemase gene was constructed. The LM alanine racemase (dal) gene (forward primer: 5′-CCA TGG TGA CAG GCT GGC ATC-3′; SEQ ID NO: 8) (reverse primer: 5′-GCT AGC CTA ATG GAT GTA TTT TCT AGG-3′; SEQ ID NO: 9) and a minimal p60 promoter sequence (forward primer: 5′-TTA ATT AAC AAA TAG TTG GTA TAG TCC-3′; SEQ ID No: 22) (reverse primer: 5′-GAC GAT GCC AGC CTG TCA CCA TGG AAA ACT CCT CTC-3′; SEQ ID No: 23) were isolated by PCR amplification from the genome of LM strain 10403S. The primers introduced a PacI site upstream of the p60 sequence, an NheI site downstream of the alanine racemase sequence (restriction sites in bold type), and an overlapping alanine racemase sequence (the first 18 bp) downstream of the p60 promoter for subsequent fusion of p60 and alanine racemase by splice overlap extension (SOE)-PCR. The sequence of the truncated p60 promoter was:

CAAATAGTTGGTATAGTCCTCTTTAGCCTTTGGAGTATTATCTCATCATT
TGTTTTTTAGGTGAAAACTGGGTAAACTTAGTATTATCAATATAAAATTA
ATTCTCAAATACTTAATTACGTACTGGGATTTTCTGAAAAAAGAGAGGAG
TTTTCC
(SEQ ID NO:7, Kohler et al, J Bacteriol
173:4668-74, 1991).

Kohler et al, J Bacteriol 173: 4668-74, 1991). Using SOE-PCR, the p60 and alanine racemase PCR products were fused and cloned into cloning vector pCR2.1 (Invitrogen, La Jolla, Calif.).
Removal of Antibiotic Resistance Genes from pGG55

The subsequent cloning strategy for removing the Chloramphenicol acetyltransferase (CAT) genes from pGG55 and introducing the p60-alanine racemase cassette also intermittently resulted in the removal of the gram-positive replication region (oriRep; Brantl et al, Nucleic Acid Res 18: 4783-4790, 1990). In order to re-introduce the gram-positive oriRep, the oriRep was PCR-amplified from pGG55, using a 5′-primer that added a NarI/EheI site upstream of the sequence (GGCGCCACTAACTCAACGCTAGTAG, SEQ ID NO: 10) and a 3′-primer that added a NheI site downstream of the sequence (GCTAGCCAGCAAAGAAAAACAAACACG, SEQ ID NO: 11). The PCR product was cloned into cloning vector pCR2.1 and sequence verified.

In order to incorporate the p60-alanine racemase sequence into the pGG55 vector, the p60-alanine racemase expression cassette was excised from pCR-p60 alanine racemase by PacI/NheI double digestion. The replication region for gram-positive bacteria in pGG55 was amplified from pCR-oriRep by PCR (primer 1, 5′-GTC GAC GGT CAC CGG CGC CAC TAA CTC AAC GCT AGT AG-3′; SEQ ID No: 20); (primer 2, 5′-TTA ATT AAG CTA GCC AGC AAA GAA AAA CAA ACA CG-3′; SEQ ID No: 21) to introduce additional restriction sites for EheI and NheI. The PCR product was ligated into pCR2.1-TOPO (Invitrogen, Carlsbad, Calif.), and the sequence was verified. The replication region was excised by EheI/NheI digestion, and vector pGG55 was double digested with EheI and NheI, removing both CAT genes from the plasmid simultaneously. The two inserts, p60-alanine racemase and oriRep, and the pGG55 fragment were ligated together, yielding pTV3.

Preparation of DNA for Real-Time PCR

Total DNA was prepared using the Masterpure Total DNA kit (Epicentre, Madison, Wis.). Briefly, bacteria were cultured for 24 hours at 37° C. and shaken at 250 rpm in 25 ml of Luria-Bertoni broth (LB). Bacterial cells were pelleted by centrifugation, resuspended in PBS supplemented with 5 mg/ml of lysozyme and incubated for 20 minutes at 37° C., after which DNA was isolated.

In order to obtain standard target DNA for real-time PCR, the LLO-E7 gene was PCR amplified from pGG55 (5′-ATGAAAAAAATAATGCTAGTTTTTATTAC-3′ (SEQ ID NO: 12); 5′-GCGGCCGCTTAATGATGATGATGATGATGTGGTTTCTG AGAACAGATG-3′ (SEQ ID NO: 13)) and cloned into vector pETblue1 (Novagen, San Diego, Calif.). Similarly, the plcA amplicon was cloned into pCR2.1. E. coli were transformed with pET-LLOE7 and pCR-plcA, respectively, and purified plasmid DNA was prepared for use in real-time PCR.

Real-Time PCR

Taqman primer-probe sets (Applied Biosystems, Foster City, Calif.) were designed using the ABI PrimerExpress software (Applied Biosystems) with E7 as a plasmid target, using the following primers: 5′-GCAAGTGTGACTCTACGCTTCG-3′ (SEQ ID NO: 14); 5′-TGCCCATTAACAGGTCTTCCA-3′ (SEQ ID NO: 15); 5′-FAM-TGCGTA CAAAGCACACACGTAGACATTCGTAC-TAMRA-3′ (SEQ ID NO: 16) and the one-copy gene plcA (TGACATCGTTTGTGTTTGAGCTAG -3′ (SEQ ID NO: 17), 5′-GCAGCGCTCTCTATACCAGGTAC-3′ (SEQ ID NO: 18); 5′-TET-TTAATGTCCATGTTA TGTCTCCGTTATAGCTCATCGTA-TAMRA-3′; SEQ ID NO: 19) as a genome target.

0.4 μM primer and 0.05 mM probe were mixed with PuRE Taq RTG PCR beads (Amersham, Piscataway, N.J.) as recommended by the manufacturer. Standard curves were prepared for each target with purified plasmid DNA, pET-LLOE7 and pCR-plcA (internal standard) and used to calculate gene copy numbers in unknown samples. Mean ratios of E7 copies/plcA copies were calculated based on the standard curves. All samples were run in triplicate in each qPCR assay which was repeated three times. Variation between samples was analyzed by Two-Way ANOVA using the KyPlot software. Results were deemed statistically significant if p<0.05.

Growth Measurements

Bacteria were grown at 37° C., 250 rpm shaking in Luria Bertani (LB) Medium +/−100 micrograms (μg)/ml D-alanine and/or 37 μg/ml chloramphenicol. The starting inoculum was calibrated based on OD₆₀₀ nm measurements to be the same for all strains.

### Results

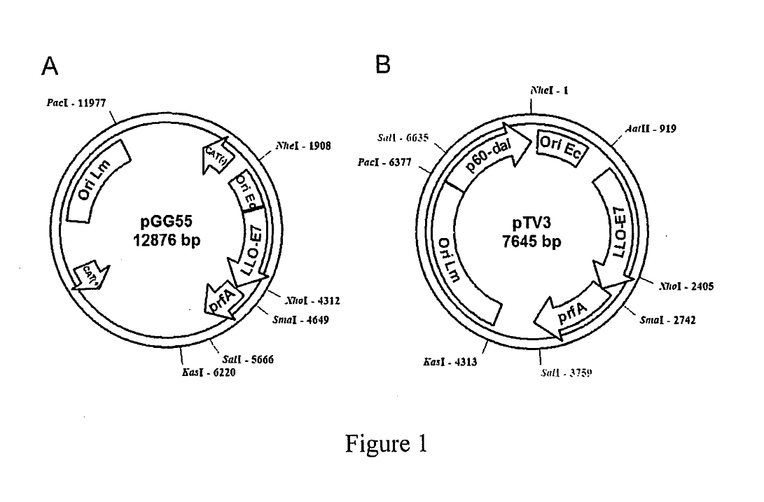

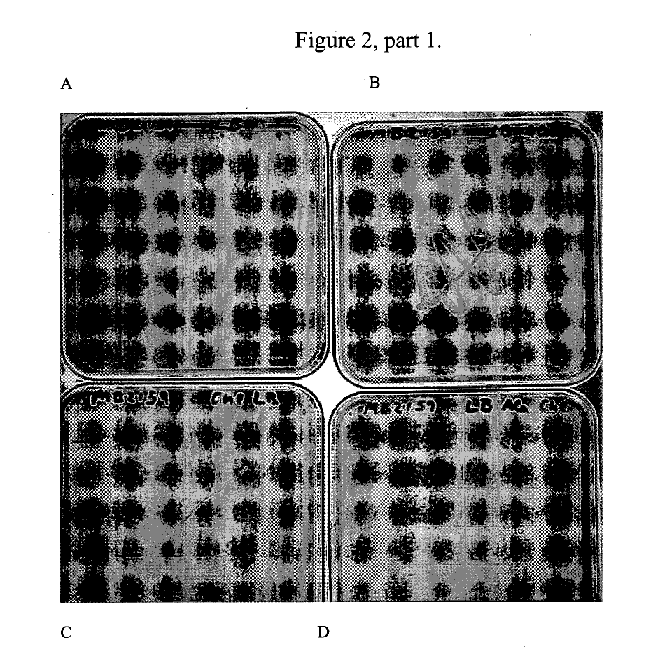

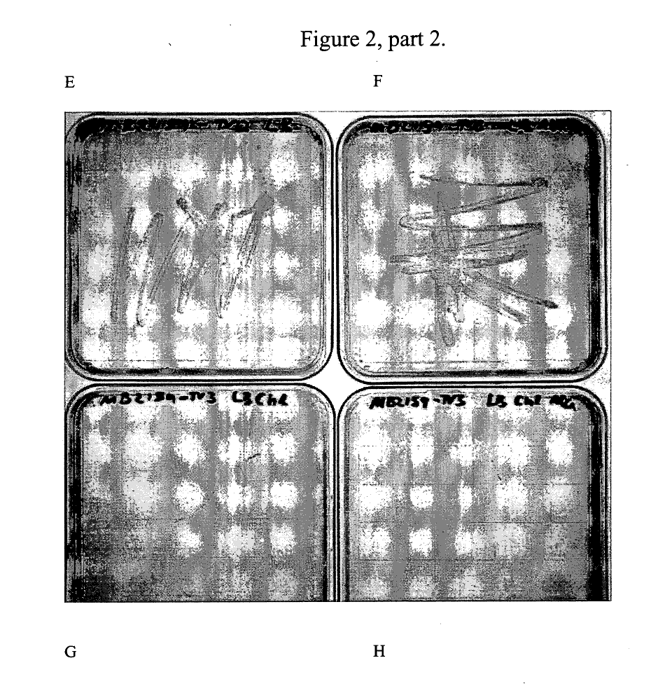

An auxotroph complementation system based on D-alanine racemase was utilized to mediate plasmid retention in E. coli without the use of an antibiotic resistance gene. E. coli strain MB2159 is an alr (−)/dadX (−) deficient mutant that is not able to synthesize D-alanine racemase. MB2159 required exogenous D-alanine for growth, but D-alanine racemase functions could be restored when expressing the alanine racemase gene from the plasmid. Plasmid pGG55, which is based on E. coli-Listeria shuttle vector pAM401, was modified by removing both CAT genes and replacing them with a p60-alanine racemase expression cassette, as described in the Methods section (FIG. 1) The resulting plasmid, pTV3, was stably maintained in E. coli. Bacterial growth on LB media that was not supplemented with additional D-alanine indicated that the alanine racemase expression cassette was active in the bacteria E. coli-pTV3 remained sensitive to chloramphenicol, indicating the successful removal of both CAT genes from the plasmid (FIG. 2).

The pTV3 copy number per cell was compared between Lm-LLOE7 in the presence of chloramphenicol and Lmdd-TV3 in the absence of chloramphenicol by real-time PCR of the E7 sequences. Lm-LLOE7 expresses LLO/E7 fusion protein from pGG55. Plasmid copy numbers of Lmdd-TV3 and Lm-LLOE7 did not significantly differ from one another, showing stable retention of plasmid pTV3.

Thus, selection for transformed bacteria can be accomplished without antibiotic resistance.

### Example 2

### Purification of a Plasmid Containing an Amino Acid Metabolism Enzyme for Use as a DNA Vaccine

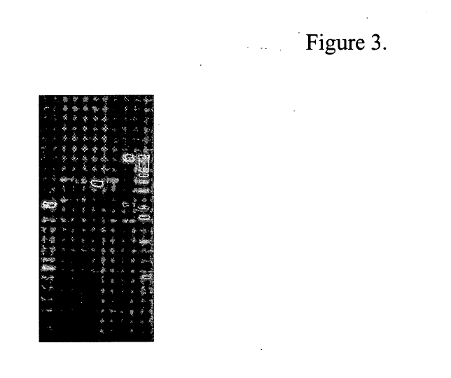

The auxotrophic bacteria transformed with the plasmid DNA vaccine pTV3 were lysed, and the plasmid DNA was isolated and purified using standard methods. Plasmids were purified using Qiagen plasmid mega kits (Qiagen Sciences, Maryland). DNA concentration was determined by the absorbance measured at 260 nm. The presence of the insert was confirmed by restriction enzyme digestion and gel electrophoresis. The plasmid DNA vaccine was restriction digested, run on an agarose gel, and stained with ethidium bromide (FIG. 3), showing the successful isolation of the plasmid.

Thus, a DNA plasmid without an antibiotic resistance gene can be propagated in an auxotrophic bacteria and isolated for use as a plasmid DNA vaccine.

### Example 3

### DNA Vaccines Carrying Plasmids Containing a Metabolic Enzyme Mediate Antigen Expression

Antigen expression from the metabolic enzyme-containing plasmid was tested in vitro by Western blot, by transforming an auxotrophic LM strains (Lmdd), using an plasmid containing an antibiotic resistance gene for comparison. When analyzing equal amounts of total protein from bacterial culture supernatants, Lmdd-TV3 cultures contained approximately double the amount of total antigen than Lm-LLOE7 cultures. This difference may be a result of a higher overall metabolic load in Lm-LLOE7, due to the larger size of the plasmid (12.8 kB) compared to Lmdd-TV3 (7.6 kB).

Thus, plasmids containing metabolic enzymes instead of antibiotic resistance genes are efficacious vehicles for expression of heterologous proteins, and thus have utility in DNA vaccines.

### Example 4

### Creation of a General Shuttle Vector Based on pTV3

pTV3 is digested with KasI or EheI and AatII, removing the prfA gene, the LLO-E7 fusion gene, and most of the LLO promoter. A multiple cloning site consisting of BamHI, XhoI, XbaI, NotI, SpeI, SmaI, and SacI is introduced by ligating the following paired oligonucleotides to the vector backbone:

5′-CGG ATC CCT CGA GCT CAG AGC GGC CGC ACT AGT CCC GGG GAG CTC G (SEQ ID No:24).

5′-TCG ACG AGC TCC CCG GGA CTA GTG CGG CCG CTC TGA GCT CGA GGG ATC CGA CGT (SEQ ID No: 25; overhanging ends that are compatible with the vector sites restricted with AatI and SalI are in italics).

An antigen cassette of interest is then ligated into the multiple cloning site. The plasmid is then used in a DNA vaccine.

### Example 5

### Creation of a General Shuttle Vector Based on an Expression Plasmid

The p60-alanine racemase expression cassette (Example 1) is introduced into an expression plasmid. For example, a commercial plasmid (e.g. pCR2.1) may be used. Subsequently, the antibiotic resistance genes are removal from the plasmid. The plasmid is then used in a DNA vaccine.

### Example 6

### In Vivo Testing of DNA Vaccines of the Present Invention Materials and Experimental Methods

Mice

Six to eight week-old C57BL/6 mice are purchased from Charles River Laboratories (Wilmington, Mass.).

Plasmid Purification

Plasmids are purified by Puresin Inc. (Malvern, Pa.). DNA concentration was determined by the A260. Plasmid identity is confirmed by restriction enzyme digestion and gel electrophoresis.

Tumor Rejection Studies

TC-1 cells are injected into C57BL/6 mice subcutaneously at a dose of 2×10⁴ cells/mouse in the left flank. Three days and 10 days later, mice are injected intramuscularly with 50 μg of plasmid(s).

### Results

The anti-tumor efficacy of DNA vaccines of the present invention is determined by measuring tumor regression in response to administration of the vaccines, as described in the Materials and Experimental Methods section. Alternatively, prevention of tumor growth is utilized as a model; in this, case, the DNA vaccines are administered prior to tumor implantation.

In other experiments, one or more cytokine plasmids (e.g. plasmids encoding GM-CSF or MIP-1α) is mixed with the E7 plasmids. Tumor regression and/or prevention of tumor formation is assessed.

DNA vaccines of the present invention are found to both prevention tumor formation and induce regression of existing tumors. Thus, DNA vaccines of the present invention have utility in inducing therapeutic and prophylactic immune responses.

### Example 7

### Further in Vivo of DNA Vaccines of the Present Invention

### Material and Experimental Methods

Three-color flow cytometry for CD8 (53-6.7, FITC conjugated), CD62 ligand (CD62L; MEL-14, APC conjugated), and PE conjugated-E7 H-2Db tetramer is performed using a FACSCalibur flow cytometer. Splenocytes are stained at room temperature with H-2Db tetramers loaded with the E7 peptide (RAHYNIVTF). Tetramers are provided by the National Institute of Allergy and Infectious Diseases Tetramer Core Facility and were used at a 1:200 dilution. The CD8⁺, CD62L^{low} subset is selected, and percentages of tetramer⁺ cells are compared using FlowJo software (Tree Star, Inc, Ashland, Oreg.).

### RESULTS

Next, the ability of the DNA vaccines of the present invention to induce antigen-specific T cells (e.g. E7-specific CD8⁺ T cells) is determined. Mice are immunized and boosted with E7 or ΔLLO-E-7 plasmid plus the plasmids encoding GM-CSF and MIP-1α. 9 days later after the second immunization, splenocytes are isolated and stained with H-2Db tetramers loaded with the E7 peptide. DNA vaccines of the present invention are found to induce significant percentages of antigen-specific T cells. These results confirm the ability of E7 DNA vaccines to elicit anti-E7 CD8⁺ T cell responses.

What is claimed is:

1. A DNA vaccine for generating an immune response against a protein antigen, said DNA vaccine comprising an antibiotic resistance gene-free plasmid, said antibiotic resistance gene-free plasmid comprising:
a first nucleic acid sequence encoding a polypeptide that comprises said protein antigen; and
a second nucleic acid sequence encoding a metabolic enzyme,
wherein said antibiotic resistance gene-free plasmid is grown in an auxotrophic bacterial strain, whereby said metabolic enzyme complements a metabolic deficiency of said auxotrophic bacterial strain, thereby generating an immune response against a protein antigen.

2. The DNA vaccine of claim 1, wherein said antibiotic resistance gene-free plasmid further comprises a transcription factor.

3. The DNA vaccine of claim 2, wherein said transcription factor is lacking in a chromosome of said auxotrophic bacterial strain.

4. The DNA vaccine of claim 1, wherein said polypeptide is a fusion protein comprising said protein antigen and an additional polypeptide, wherein said additional polypeptide is a non-hemolytic fragment of an LLO protein, a PEST-like amino acid sequence, or an ActA protein.

5. The DNA vaccine of claim 1, wherein said first nucleic acid sequence is operably linked to a promoter/regulatory sequence.

6. The DNA vaccine of claim 1, wherein said second nucleic acid sequence is operably linked to a promoter/regulatory sequence.

7. The DNA vaccine of claim 1, wherein said metabolic enzyme is an amino acid metabolism enzyme.

8. The DNA vaccine of claim 1, wherein said metabolic enzyme is an alanine racemase enzyme.

9. The DNA vaccine of claim 1, wherein said metabolic enzyme is a D-amino acid transferase enzyme.

10. The DNA vaccine of claim 1, wherein said auxotrophic bacterial strain is an auxotrophic E. coli strain.

11. The DNA vaccine of claim 1, wherein said DNA vaccine further comprises an adjuvant, cytokine-encoding nucleotide molecule, or pharmaceutically acceptable carrier

12. A method for treating a disease agent, wherein said disease agent expresses the protein antigen of claim 1, comprising administering the DNA vaccine of claim 1.

13. The method of claim 12, wherein said disease agent is a pathogen.

14. The method of claim 12, wherein said disease agent is a cancer cell or neoplastic cell.

15. A method of preparing a DNA vaccine for generating an immune response against a protein antigen, said method comprising
a. growing an auxotrophic bacterial strain containing a plasmid, wherein said plasmid comprises:
first nucleic acid sequence encoding a polypeptide that comprises said protein antigen; and
a second nucleic acid sequence encoding a metabolic enzyme, whereby said metabolic enzyme complements a metabolic deficiency of said auxotrophic bacterial strain;
and wherein said plasmid does not contain an antibiotic resistance gene; and
b. and isolating the plasmid DNA vaccine from said auxotrophic bacterial strain,
thereby preparing a DNA vaccine for generating an immune response against a protein antigen.

16. The method of claim 15, whereby said plasmid does not confer antibiotic resistance to said auxotrophic bacterial strain.

17. The method of claim 15, wherein said auxotrophic bacterial strain further lacks a transcription factor.

18. The DNA vaccine of claim 17, wherein said transcription factor is lacking in a chromosome of said auxotrophic bacterial strain.

19. The method of claim 15, wherein said polypeptide is a fusion protein comprising said protein antigen and an additional polypeptide, wherein said additional polypeptide is a non-hemolytic fragment of an LLO protein, a PEST-like amino acid sequence, or an ActA protein.

20. The method of claim 15, wherein said first nucleic acid sequence is operably linked to a promoter/regulatory sequence.

21. The method of claim 15, wherein said metabolic enzyme is an amino acid metabolism enzyme.

22. The method of claim 15, wherein said metabolic enzyme is an alanine racemase enzyme.

23. The method of claim 15, wherein said metabolic enzyme is a D-amino acid transferase enzyme.

24. The method of claim 15, wherein said second nucleic acid sequence is operably linked to a promoter/regulatory sequence.

25. The method of claim 15, further comprising mixing said plasmid with an adjuvant, cytokine-encoding nucleotide molecule, or pharmaceutically acceptable carrier.

26. The method of claim 15, wherein said auxotrophic bacterial strain is an auxotrophic E coli strain.

27. The method of claim 15, further comprising contacting said auxotrophic bacterial strain with a plasmid, whereby said auxotrophic bacterial strain takes up said plasmid.

28. A method for treating a disease agent, wherein said disease agent expresses the protein antigen of claim 15, comprising administering a DNA vaccine prepared by the method of method of claim 15.

29. The method of claim 28, wherein said disease agent is a pathogen.

30. The method of claim 28, wherein said disease agent is a cancer cell or neoplastic cell.
